(19) United States
(12) Plant Patent Application Publication
(10) Pub. No.: US 2012/0096611 P1
(43) Pub. Date: Apr. 19, 2012

(54) Late harvest avocado tree

(50) ;

(76) Inventor: Charlie Dorsey, Homestead, FL (US)

(21) Appl. No.: 12/925,213

(22) Filed: Oct. 18, 2010

(51) Int. Cl. A01H 5/00

(52) U.S. Cl. PLT/200

(57) ABSTRACT

A new avocado tree has an intermediate growth pattern, with fruit that mature from the end of March through mid-May. The fruit are found in a diversity of shapes, and large lenticles are found on the skin as the fruit ripens.

### FIELD OF THE PLANT

This plant patent pertains to the Persea Americana, or Avocado.

### BACKGROUND OF THE VARIETY

The present invention relates to a new and distinct variety of avocado tree which is characterized by its ability to have two fruit pickings, of good eating quality and relatively late picking and more particularly characterized by the fruit being easily stored on the tree.

The avocado season in Florida, and especially south Florida in Dade and Broward counties, runs from late May of this year, 2010, till March of next year 2011. A relatively small amount of avocados are available for the marketplace from Mid-March of the year following through late May of next year.

The popularity of Mexican food is ever increasing and as such the market for Florida avocados has been expanding at a rapid pace, throughout the calendar year. The variety of this invention has been named Buck 3, as a tribute to the Inventor's son, whose nickname was Buck, has a late maturation of the fruit, later than either the Alfa or Wheeling varieties.

### ORIGIN AND ASEXUAL REPRODUCTION OF THE VARIETY

The original tree was found as a sport on Applicant's property in Dade County south of Miami. It was asexually reproduced in the 2005-2006 year by the grafting of bud stock from the parent tree onto Walden root stock and named for applicant's son.

### SUMMARY OF THE NEW VARIETY

The original tree and the asexually reproduced progeny are moderately large in both width—spread—and height. It is characterized further by its Type B flowers and excellent fruit set. The growth pattern of the tree is intermediate. The fruit mature from the end of March through mid-May when very few avocados are available in the marketplace in South Florida. The fruit is seen to have a diversity of shapes.

### BRIEF DESCRIPTION OF THE FIGURES

FIG. 1 is a color photograph showing the dark green upper surface of the leaves of this avocado tree and the light green under surface of various shaped leaves of the avocado tree of this application.

FIG. 2 is a color photograph showing the diversity of shapes of the avocado tree of this patent application.

FIG. 3 is a color photograph of the skin of the fruit showing the large lenticles found on the skin as the fruit ripens.

FIG. 4 is a closeup color photograph showing the pedicel size, shape and insertion.

FIG. 5A and FIG. 5B are color photographs of the two fruits from the same tree, showing the color of the flesh and two different shape seeds.

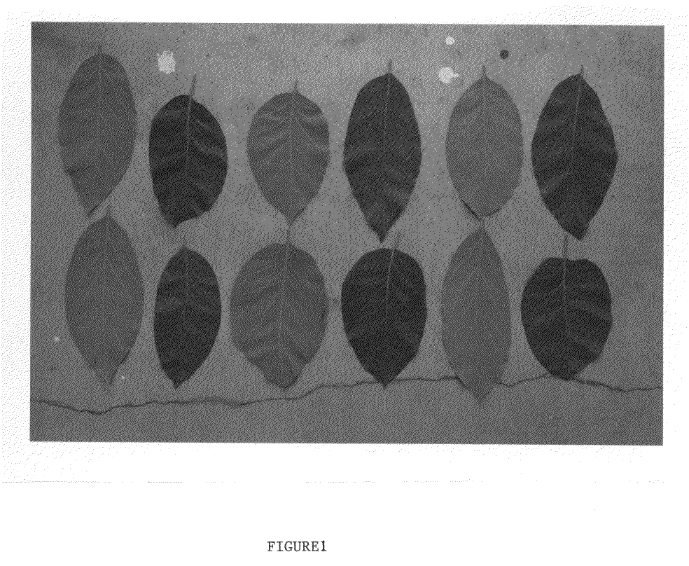

### DETAILED DESCRIPTION OF THE VARIETY

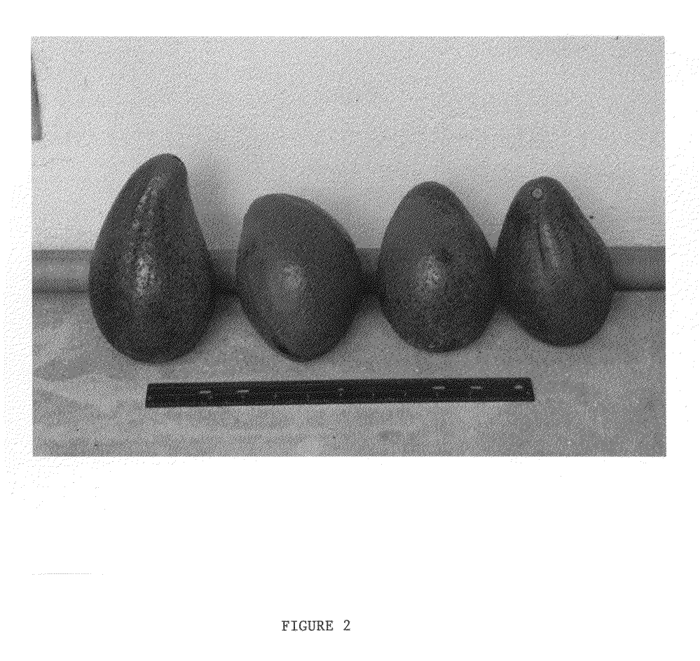

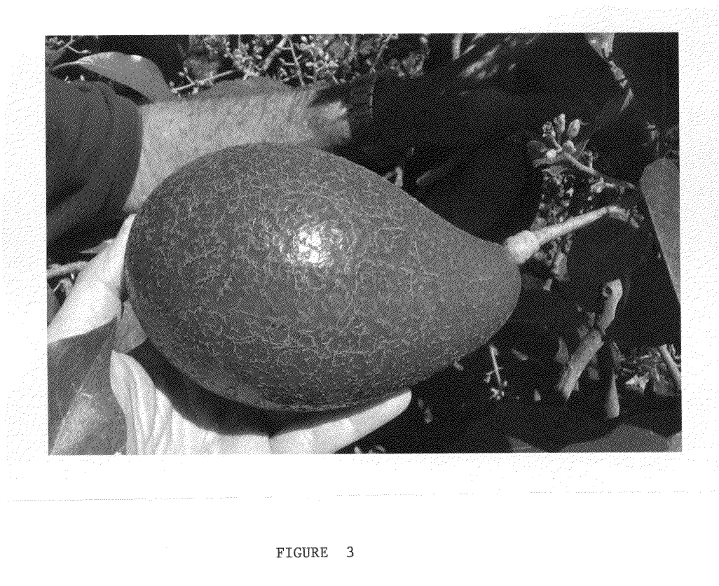

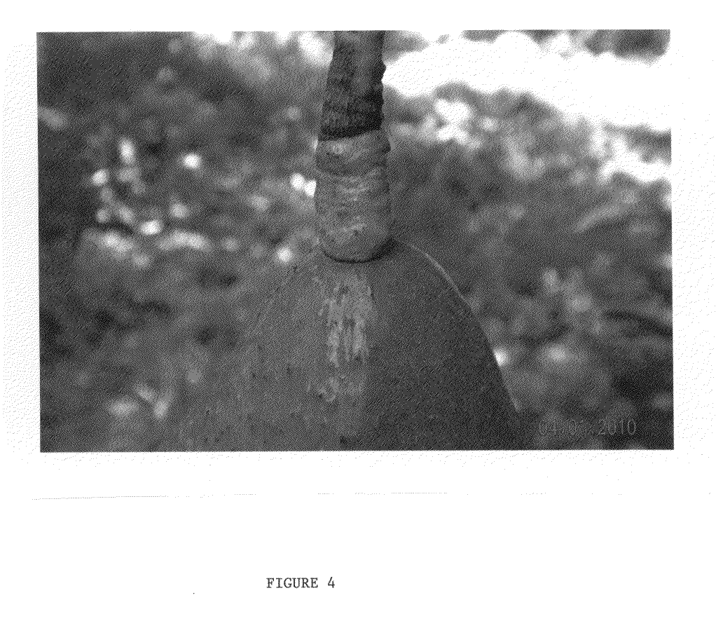

Reference is now made to the botanical details of the new and distinct variety of avocado tree. Major color code plate identification are made by reference to the A. Maerz and M. Aul Dictionary of color published by McGraw Hill Company, Second Edition, 1950 , as well as common accepted color terminology.
Tree: The seedling tree is moderately large, broad and tall. The growth pattern is intermediate or spreading.
Leaves:
   Size and shape.—Fairly large, resembling Guatemalan type or hybrid leaves Leaves are ovate and broad with a round base and pointed tips. The leaf base is acute to obtuse, leaf tip acute; pubescence, sparse. Leaf blades are of an average length of 22.2 cm (8 12/16 inch) and width of 11.8 cm (4 10/16 inch). The length/width ratio averages 1.9.
   Venation.—The leaf margins are entire with 13 to 16 primary veins. The venation relief on the upper leaf surface is intermediately raised. Leaf texture is semi-hard.
   Petioles.—3.3 to 4.3 cm (1.3 to 1.7 inch) long. The upper surface leaf color ranges from stock green to forest green or 003333 to 003300 (Coutsoukis, 2004) whereas the lower surface color ranges from odd green to seas spray (Hall Color Charts, 2004) or 336600 to 336633 (Coutsoukis, 2004). The leaf petiole and main veins are canary yellow (Hall Color Charts, 2004) or 669900 (Coutsoukis, 2004).
Flowers: Type B, stamens which open in the morning. The bloom season is late February to early April.
Fruit:
   Shape.—obovate, with a small flattened area toward the apex on one side. See FIG. 2. fruit. As fruit ripen the lenticels become more pronounced (brown color) and peel color darkens. Fruit quality is excellent with buttery, smooth flesh with an excellent nutty flavor.
   Stem insertion and shape.—The pedicel is inserted asymmetrically and is mostly conical, with some being cylindrical See FIG. 4.
   Size.—Medium to medium large, average fruit weight is 526.7 g (18.6 oz) with a range of about 262 to 668 g (9.3 to 23.6 oz).

WEIGHT AND SIZING TABLE

| | Metric units | | English units | | | |
|---|---|---|---|---|---|---|
| Parameter | Average/Range | | Average | | Range | |
| Whole fruit | 526.7 g/262.1-668.1 | g | 18.6 | oz | 9.3-23.6 | oz |
| Pulp | 247.0 g/189.1-503.1 | g | 8.7 | oz | 6.7-17.8 | oz |
| Seed | 73.6 g/44.8-123.1 | g | 2.6 | oz | 1.6-4.3 | oz |
| Peel | 50.4 g/29.3-60.9 | g | 1.8 | oz | 1.0-2.2 | oz |
| Fruit diameter | 9.1 cm/8.2-9.9 | cm | 3 9/16 | inch | 3 3/16-3 14/16 | inch |
| Peel thickness | 1.7 | mm | 1.3-3.0 | mm | 0.8/16- 2/16 | inch |

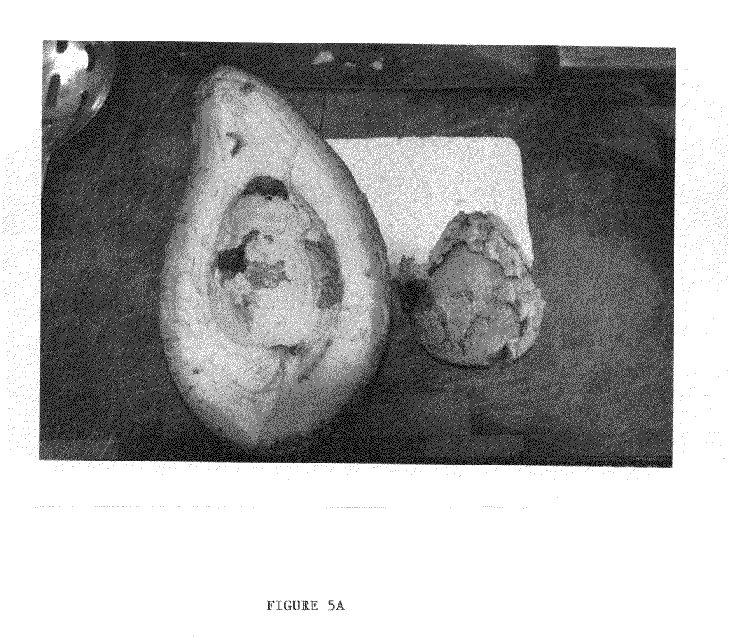

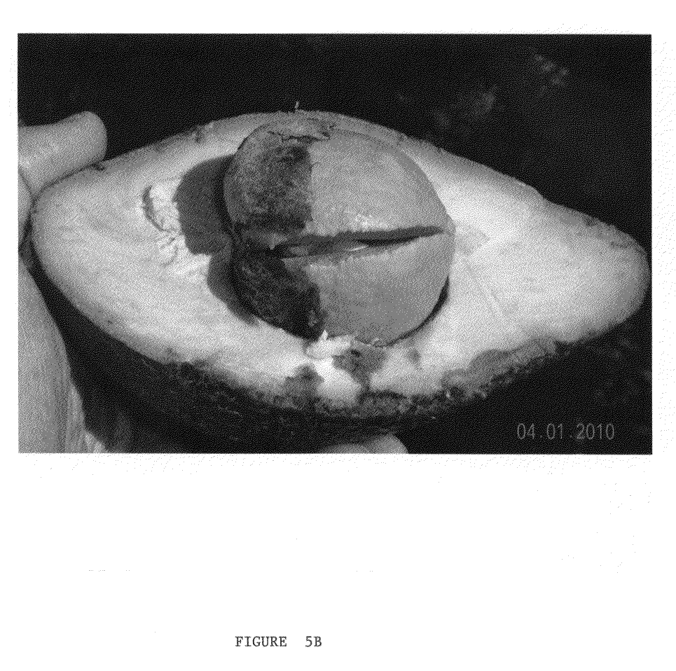

Peel.—average peel weight is 50.4 g (1.8 oz) average peel thickness is 1.7 mm ( 1/16 inch) peel is stock green to forest green (green to dark green) (Hall Color Charts, 2004) or 336600 to 003300 (Coutsoukis, 2004); slightly rough but smooth on the flattened area located toward the base (stylar end) of the fruit. A longitudinal lettuce colored (Hall Color Charts, 2004) or 66CC00 to 66CC33 (Coutsoukis, 2004) streak may be present on most fruit.
Pulp.—averages about 46.9%. ratio of pulp weight to seed weight is 3.4. Pulp weight average is 247 g (8.7 oz). thickness generally medium, however in some fruit a little less than medium and ranges from 2.86 to 3.7 cm (1 2/16- 1 7/16 inch). color — dark green thin ring about 6 mm (¼ inch) adjacent to the peel and pulp color near seed is deep yellow. See FIG. 5. consistency — smooth and buttery without fibers.
Seed.—medium size, rounded to pear shaped (range 44.9-123.1 g; average 2.6 oz) The seed is tight (not loose) in the cavity and average seed weight is 14% of total fruit weight. Seed coat color is brown per FIG. 5 and does not adhere to pulp early in the season but may adhere later in the season.
Harvest and production: Mature fruit may be harvested from late March to mid-May. On average the time from harvest to ripening (softening) at room temperature (˜24° C.; 75° F.) took 8.5 days with a range of 5 to 14 days. The Buck 3 avocado tree bears every year but one year has a very heavy crop and the other a lighter crop, the lighter one being about 37.4 kg (82.5 lbs; 1.5 bushels)

Because of the outstanding and unique horticultural characters, especially its very late harvest season, this cultivar can be planted to extend Florida's avocado season. Although this new variety of avocado possesses the above described characteristics, it is to be understood that variations and fluctuations may occur in the magnitude and characteristics thereof due to changes in growing and climate conditions, irrigation and fertilization pruning and pest control.

What is claimed is:

1. A new and distinct variety of avocado substantially as illustrated and described which is characterized by its late harvest season, variation in stone shape, and fine eating quality with non-fibrous flesh.
